(19) United States
(12) Patent Application Publication
(10) Pub. No.: US 2024/0415962 A1
(43) Pub. Date: Dec. 19, 2024

(54) METHOD FOR SITE-DIRECTED INTEGRATION OF TARGET GENE INTO SPECIFIC SITE OF IMMUNE CELL AND USE THEREOF

(71) Applicant: XI'AN YUFAN BIOTECHNOLOGIES CO., LTD., Xi'an, Shaanxi (CN)

(72) Inventors: Fei LONG, Xi'an, Shaanxi (CN); Sai LI, Xi'an, Shaanxi (CN); Liqin ZHANG, Xi'an, Shaanxi (CN)

(21) Appl. No.: 18/701,166

(22) PCT Filed: Oct. 18, 2022
(86) PCT No.: PCT/CN2022/125908

§ 371 Date: Apr. 12, 2024

(30) Foreign Application Priority Data
Oct. 18, 2021 (CN) 202111208589.0

(51) Int. Cl. A61K 39/00; C07K 14/725; C07K 16/28; C12N 9/22; C12N 15/11; C12N 15/90

(52) U.S. Cl. CPC A61K 39/4611; A61K 39/4631; A61K 39/464412; C07K 14/7051; C07K 16/2803; C12N 9/22; C12N 15/11; C12N 15/907; C12N 2310/20; C12N 2800/80

(57) ABSTRACT

A method for the site-directed integration of a target gene into a specific site of an immune cell and a use thereof, in particular being a method for the site-directed integration of a target gene (such as a CAR gene) into a specific site of an immune cell (such as HPK1) based on a non-viral vector, and a use thereof. The method completes gene knockout and target gene introduction in one step, and prepares a cell that has undergone gene knockout at a specific site and that stably expresses a target gene. Moreover, the method has the characteristics of simple preparation, low costs, stable expression, and stable function, and does not affect the gene characteristics of a cell. In addition, the obtained cell has high killing efficiency and strong infiltration capability.

### TECHNICAL FIELD

The present invention relates to the technical field of biomedicine, and particularly to a method for site-specific integration of a target gene (such as a CAR gene) into a specific site (such as HPK1) of an immune cell based on a non-virus vector and use thereof.

### BACKGROUND

Gene therapy refers to the introduction of foreign genes into target cells to correct or compensate for diseases caused by defective and abnormal genes, so as to achieve the therapeutic goal. Gene therapy is primarily the treatment of diseases that pose a serious threat to human health, including: malignant tumors, genetic diseases (such as hemophilia, congenital amaurosis, and cystic fibrosis), cardiovascular diseases, infectious diseases (such as AIDS, and the like), autoimmune diseases (such as rheumatoid arthritis, ankylosing spondylitis, and psoriasis, and the like), and the like.

Tumor is one of the main diseases threatening human health, and immunotherapy against tumors has become a research hotspot for tumor treatment in recent years. The tumor immune cell therapy is to modify, culture and amplify immune cells collected from human body in vitro, and subsequently to reinfuse the cells into the body of a patient to stimulate and enhance the autoimmune function of the body, thereby achieving the effects of inhibiting tumor growth and killing tumor cells.

Chimeric antigen receptor T cell (CART cell) immunotherapy has achieved significant success in the treatment of hematological tumors. CAR-bearing T cells can directly recognize a target antigen, regardless of whether the antigen is presented on the MHC, thereby avoiding “MHC restriction”. A stimulation by the target antigen in the body can induce a series of cell-mediated immune responses, finally killing target cells. Traditional CART therapy is to transfer CAR into T cells in the form of virus vectors by using genetic engineering technology, and this strategy often requires infection with virus vectors such as lentiviruses. In one aspect, virus vectors are expensive to produce and relatively complex in process. In another aspect, they may pose the safety risk of random insertion. For example, there is a risk of insertion point mutations and the induction of cells resulting in transformation, even carcinogenesis.

In gene therapy, immune checkpoints play a very important role. There are now more and more attempts to introduce foreign genes and knock out immune checkpoint genes in cells. In addition, in the preparation of universal cell therapy products (e.g., UCART), it is also necessary to simultaneously knock out the TCR gene. The existing conventional method is to carry out the introduction of foreign genes and gene knockout step by step. Based on this, some site-specific integration technologies are disclosed in the prior art. For example, patent application CN105524943A discloses a method for site-specific integration of CAR genes into the T cell AAVS1 site based on double-stranded microvectors, and patent application CN111944848A discloses a method for improving site-specific integration of genes into cells based on electroporation technology and CRISPER-Cas9 technology. Patent application CN113005092A discloses the preparation of CART cells targeting LMP1 with knockout of PD1, using a combination treatment of CART cells targeting LMP1 in combination with knockout of PD1. However, the methods and homologous recombination repair templates adopted in the prior art, such as conventional plasmids, and single-stranded and double-stranded DNA, often have the defects of complicated method steps, low CAR transduction efficiency, and the like.

### SUMMARY

In order to overcome the defects of the prior art, the present invention provides a method for site-specific integration of a target gene (such as a CAR gene) into a specific site (such as HPK1) of an immune cell based on a non-virus vector, which can efficiently knock out the specific gene site and complete target gene introduction in one step, thereby simplifying the process flow, reducing the production cost and shortening the preparation time.

In a first aspect of the present invention, provided is a method for site-specific integration of a target gene into a specific site of an immune cell, which comprises the following steps:
S1, constructing a vector comprising a homologous recombination repair template;
S2, introducing a gene editing system and the vector obtained in step S1 into an immune cell together; optionally, S3, culturing and identifying the immune cell obtained in step S2.

The vector in step S1 is selected from: adeno-associated virus (AAV), minicircle DNA (mcDNA), double-stranded DNA (dsDNA), or single-stranded DNA (ssDNA), particularly minicircle DNA or single-stranded DNA.

Specifically, the homologous recombination repair template in step S1 comprises a homology arm and a target gene, and more specifically, the homologous recombination repair template comprises 1):

The minicircle DNA is from 5′ to 3′ in sequence: a target sequence (TSF), a left homology arm (LHA), a promoter, a target gene, a polyA, a right homology arm (RHA), a target sequence (TSF).

Or 2):

The single-stranded DNA is from 5′ to 3′ in sequence:
Sequence A. a gRNA target region, a left homology arm (LHA), a promoter, a target gene, a polyA, a right homology arm (RHA), a gRNA target region.
Sequence B. a gRNA non-target region, a left homology arm (LHA), a promoter, a target gene, a polyA, a right homology arm (RHA), a gRNA target region, or a gRNA target region, a left homology arm (LHA), a promoter, a target gene, a polyA, a right homology arm (RHA), a gRNA non-target region.
Sequence C. The sequence A described above paring to a gRNA non-target region+an overlap region fragment. or
Sequence D. The sequence B described above paring to a gRNA target region+an overlap region fragment or/and an overlap region+a gRNA non-target region fragment.

The single-stranded DNA may comprise 0, 1, 2, 3, 4, 5, 6, 7, 8, 9 or 10 gRNA target region(s) and gRNA non-target region(s). The number of bases of the overlap region fragment may be 0-500 bp, e.g., 0, 5, 10, 15, 20, 25, 30, 50, 100, 150, 200, 250, 300, 350, 400, 450, or 500 bp. Preferably, the single-stranded DNA may be subjected to phosphorothioate modification between 1-5 bases (such as 1, 2, 3, 4, or 5 bases) at the 5′ end and/or 3′ end. Specifically, the target gene may be a CAR gene; more specifically, the target recognized by the CAR may be: ROR1, Her2, L1-CAM, CD19, CD20, CD22, CEA, hepatitis B surface antigen, folate receptor antibody, CD23, CD24, CD30, CD33, CD38, CD276, CD44, EGFR, EGP-2, EGP-4, EPHa2, ErbB2, ErbB3, ErbB4, FBP, fetal acetylcholine receptor, GD2, GD3, HMW-MAA, IL-22R-α, IL-13R-α2, kdr, k light chain, Lewis Y, L1 cell adhesion molecule (CD171), MAGE-A1, mesothelin, MUC1, MUC16, PSCA, NKG2D ligand, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGEA3, CE7, Wilms tumor 1 (WT-1), cyclin A1 (CCNA1), BCMA, and interleukin 12, or any combination thereof; in some embodiments of the present invention, the target recognized by the CAR is CD19, i.e., CD19 CAR. In one embodiment of the present invention, the CAR gene comprises the nucleotide sequence set forth in SEQ ID NO: 25, particularly the CAR gene consists of the nucleotide sequence set forth in SEQ ID NO: 25.

Specifically, the specific site may be genes such as HPK1, PD-1, or TRAC, and the like, particularly HPK1, and particularly HPK1 EXON.

Specifically, the TSF comprises or consists of the nucleotide sequence set forth in SEQ ID NO: 7 and/or 8, or a nucleotide sequence having at least 70%, 75%, 80%, 85%, 90%, 91%, 92%, 93%, 94%, 95%, 96%, 97%, 98%, or 99% identity or complementarity to the nucleotide sequence set forth in SEQ ID NO: 7 and/or 8; that is, the sequence of the TSF is set forth in SEQ ID NO: 7 and/or SEQ ID NO: 8.

In one embodiment of the present invention, the gRNA target region comprises the nucleotide sequence set forth in SEQ ID NO: 9 and/or 11, or a nucleotide sequence having at least 50%, 55%, 60%, 65%, 70%, 75%, 80%, 85%, 90%, 91%, 92%, 93%, 94%, 95%, 96%, 97%, 98%, or 99% identity to the nucleotide sequence set forth in SEQ ID NO: 9 and/or 11. Preferably, the gRNA target region is the sequence set forth in SEQ ID NO: 9 and/or 11.

In one embodiment of the present invention, the gRNA non-target region comprises the nucleotide sequence set forth in SEQ ID NO: 10 and/or 12, or a nucleotide sequence having at least 50%, 55%, 60%, 65%, 70%, 75%, 80%, 85%, 90%, 91%, 92%, 93%, 94%, 95%, 96%, 97%, 98%, or 99% identity to the nucleotide sequence set forth in SEQ ID NO: 10 and/or 12. Preferably, the gRNA non-target region is the sequence set forth in SEQ ID NO: 10 and/or 12.

Specifically, the left homology arm and the right homology arm have a length of 40-2000 bp (e.g., 40, 100, 200, 300, 400, 500, 600, 700, 800, 900, 1000, 1500, or 2000 bp), respectively; in one embodiment of the present invention, the left homology arm and the right homology arm comprise the nucleotide sequence set forth in SEQ ID NOs: 13 and 14, respectively, or are set forth in SEQ ID NOs: 13 and 14, respectively; in another embodiment of the present invention, the left homology arm and the right homology arm comprise the nucleotide sequence set forth in SEQ ID NOs: 15 and 16, respectively, or are set forth in SEQ ID NOs: 15 and 16, respectively; in another embodiment of the present invention, the left homology arm and the right homology arm comprise the nucleotide sequence set forth in SEQ ID NOs: 17 and 18, respectively, or are set forth in SEQ ID NOs: 17 and 18, respectively. Specifically, the promoter may be an EF1α promoter, a CMV promoter, an SFFV promoter, and the like. In one embodiment of the present invention, the promoter is an EF1α promoter, the nucleotide sequence thereof is set forth in SEQ ID NO: 19.

Specifically, the polyA may be BGHpA, SV40 polyA, or WPRE, the nucleotide sequences thereof are set forth in SEQ ID NOs: 20-22, respectively; in one embodiment of the present invention, the polyA is SV40 polyA.

Specifically, the gene editing system in step S2 is a gene editing system that reduces expression of a specific site (e.g., HPK1) in an immune cell, which may be a CRISPR system, ZFN, TALEN, and the like, particularly a CRISPR system, such as a CRISPR/Cas9 system, a CRISPR/Cas12a system, a CRISPR/Cas13 system, particularly a CRISPR/Cas9 system.

Specifically, the CRISPR system comprises or consists of a gRNA and a nuclease.

Specifically, the nuclease may be SpCas9, SaCas9, eSpCas9, Cas12a, Cas13, or cpf1 and mutants thereof; in some embodiments of the present invention, the nuclease may be eSpCas9.

Specifically, the gRNA specifically targets a specific site (e.g., HPK1), which may have a target domain complementary to a target sequence: in some embodiments of the present invention, the target domain sequence comprises or consists of the nucleotide sequence set forth in any one of SEQ ID NOs: 1-6.

In some embodiments of the present invention, the gRNA comprises and in particular consists of the nucleotide sequence set forth in SEQ ID NO: 23 and/or 24, or a nucleotide sequence having at least 70%, 75%, 80%, 85%, 90%, 91%, 92%, 93%, 94%, 95%, 96%, 97%, 98%, or 99% identity to the nucleotide sequence set forth in SEQ ID NO: 23 and/or 24.

Specifically, the gRNA may also comprise a chemical modification of the base, such as methylation modification or thio-modification or a combination of both; more specifically, 1-5 bases (e.g., 1, 2, 3, 4, or 5 bases) at the 5′ end and/or 3′ end of the gRNA are subjected to 2′-O-methylation modification and/or phosphorothioate modification. In some embodiments of the present invention, 3 bases at the 3′ end of the gRNA are subjected to 2′-O-methylation modification.

In some embodiments of the present invention, the gene editing system in step S2 is in the form of a composition or complex of a gRNA and a nuclease, particularly a complex of a gRNA and a nuclease, such as CRISPR-Cas9 RNP (ribonucleoprotein complex).

Specifically, the method for introducing the immune cell in step S2 may be transformation, transfection, heat shock, electroporation, transduction, microinjection, and the like; in some embodiments of the present invention, the method for introducing the immune cell is electroporation.

In some embodiments of the present invention, step S2 comprises: mixing a nuclease protein and a gRNA, incubating, adding the vector in step S1, mixing with an immune cell, electroporating.

Specifically, the condition of the electroporation may be 1300 V, 20 ms, and 1 pulse.

Specifically, the immune cell may be a T cell, an NK cell, a B cell, a macrophage, a dendritic cell, or a monocyte, particularly a T cell, such as NKT or γδT. Specifically, the T cell may be CD3 positive.

Specifically, the immune cell may be of autologous origin (e.g., from a subject suffering from a disease requiring gene therapy) or of allogeneic origin (e.g., from a healthy donor).

Specifically, the immune cell used in step S2 may be obtained by the following steps: PBMC isolation; T cell activation.

Specifically, the PBMC isolation step may comprise: diluting a peripheral blood sample, mixing with a lymphocyte isolation solution, centrifuging, pipetting cells in the buffy coat (then washing, resuspending, and cryopreserving). Specifically, the T cell activation step may comprise: activating and culturing the PBMCs by CD3/CD28 beads. Specifically, the identification in step S3 may comprise identification of a gene integration site, identification of CAR receptor expression, identification of CAR receptor immune activation function, and the like.

In a second aspect of the present invention, provided is a method for site-specific integration of a target gene into a specific site of an immune cell, wherein the method comprises introducing the target gene into the specific site of the immune cell using a gene editing system.

Specifically, the target gene may be a CAR gene; more specifically, the target recognized by the CAR may be: ROR1, Her2, L1-CAM, CD19, CD20, CD22, CEA, hepatitis B surface antigen, folate receptor antibody, CD23, CD24, CD30, CD33, CD38, CD276, CD44, EGFR, EGP-2, EGP-4, EPHa2, ErbB2, ErbB3, ErbB4, FBP, fetal acetylcholine receptor, GD2, GD3, HMW-MAA, IL-22R-α, IL-13R-α2, kdr, κ light chain, Lewis Y, L1 cell adhesion molecule (CD171), MAGE-A1, mesothelin, MUC1, MUC16, PSCA, NKG2D ligand, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGEA3, CE7, Wilms tumor 1 (WT-1), cyclin A1 (CCNA1), BCMA, and interleukin 12, or any combination thereof; in some embodiments of the present invention, the target recognized by the CAR is CD19, i.e., CD19 CAR. In one embodiment of the present invention, the CAR gene comprises the nucleotide sequence set forth in SEQ ID NO: 25. Particularly the CAR gene consists of the nucleotide sequence set forth in SEQ ID NO: 25.

Specifically, the specific site may be HPK1, PD-1, TRAC, and the like, particularly HPK1, and particularly HPK1 EXON. Specifically, the target domain sequence complementary to the target sequence at the HPK1 site (e.g., HPK1 EXON) comprises or consists of the nucleotide sequence set forth in any one of SEQ ID NOs: 1-6. Specifically, the target sequence comprises the 3′ end of SEQ ID NOs: 1-6 added with a PAM region. The PAM is typically a 2-6 base pair sequence, such as 5′-NGG-3′ (wherein N is any base), 5′-NGA-3′, 5′-YG-3′ (wherein Y is a pyrimidine), 5′TTN-3′, or 5′-YTN-3′. In specific embodiments, the added PAM may be adjusted according to the type of the nuclease.

Specifically, the gene editing system may be a CRISPR system, ZFN, TALEN, and the like, particularly a CRISPR system, such as a CRISPR/Cas9 system, a CRISPR/Cas12a system, a CRISPR/Cas13 system, particularly a CRISPR/Cas9 system.

Specifically, the CRISPR system comprises or consists of a gRNA and a nuclease.

Specifically, the nuclease may be SpCas9, SaCas9, eSpCas9, Cas12a, Cas13, or cpf1 and mutants thereof; in some embodiments of the present invention, the nuclease may be eSpCas9.

Specifically, the gRNA specifically targets a specific site (e.g., HPK1), which may have a target domain complementary to a target sequence; in some embodiments of the present invention, the target domain sequence comprises or consists of the nucleotide sequence set forth in any one of SEQ ID NOs: 1-6.

Specifically, the target gene may be introduced into a specific site of an immune cell via a vector carrying the target gene. The vector is selected from: adeno-associated virus (AAV), minicircle DNA (mcDNA), double-stranded DNA (dsDNA), or single-stranded DNA (ssDNA), particularly minicircle DNA or single-stranded DNA.

Specifically, the vector comprises a homology arm and a target gene. Preferably, further included is a target sequence, a promoter, and/or a polyA, and the like.

Specifically, the immune cell may be a T cell, an NK cell, a B cell, a macrophage, a dendritic cell, or a monocyte, particularly a T cell, such as NKT or γδT. Specifically, the T cell may be CD3 positive.

Specifically, the immune cell may be of autologous origin (e.g., from a subject suffering from a disease requiring gene therapy) or of allogeneic origin (e.g., from a healthy donor).

In a third aspect of the present invention, provided is the site-specific integrated immune cell prepared according to the first or second aspect. For example, CAR-immune cells (e.g., CART, CAR-NK, CAR-NKT, or CAR-γδT cells, particularly CART cells) with knockout of a specific site (e.g., HPK1) gene.

In some embodiments of the present invention, the cell described above is a CD19 CART cell with knockout of HPK1 gene.

Specifically, the CART cell described above may be an autologous CART cell or a universal CART cell.

In a fourth aspect of the present invention, provided is a CAR gene comprising a ligand binding domain, a transmembrane domain, and a costimulatory domain.

Specifically, the ligand binding domain may target a target selected from: ROR1, Her2, L1-CAM, CD19, CD20, CD22, CEA, hepatitis B surface antigen, folate receptor antibody, CD23, CD24, CD30, CD33, CD38, CD276, CD44, EGFR, EGP-2, EGP-4, EPHa2, ErbB2, ErbB3, ErbB4, FBP, fetal acetylcholine receptor, GD2, GD3, HMW-MAA, IL-22R-α, IL-13R-α2, kdr, κ light chain, Lewis Y, L1 cell adhesion molecule (CD171), MAGE-A1, mesothelin, MUC1, MUC16, PSCA, NKG2D ligand, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGEA3, CE7, Wilms tumor 1 (WT-1), cyclin A1 (CCNA1), BCMA, and interleukin 12, or any combination thereof; in some embodiments of the present invention, the target is CD19, i.e., CD19 CAR.

In one embodiment of the present invention, the ligand binding domain is an scFv comprising the nucleotide sequence from position 70 to position 795 of SEQ ID NO: 25.

Specifically, the transmembrane domain may be selected from transmembrane domains of the following proteins: TCRα chain, TCRβ chain, TCRγ chain, TCRδ chain, CD3ζ subunit, CD38 subunit, CD3γ subunit, CD38 subunit, CD45, CD4, CD5, CD8a, CD9, CD16, CD22, CD33, CD28, CD37, CD64, CD80, CD86, CD134, CD137, and CD154.

Specifically, the costimulatory domain may be selected from costimulatory signaling domains of the following proteins; TLR1, TLR2, TLR3, TLR4, TLR5, TLR6, TLR7, TLR8, TLR9, TLR10, CARD11, CD2, CD7, CD8, CD18 (LFA-1), CD27, CD28, CD30, CD40, CD54 (ICAM), CD83, CD134 (OX40), CD137 (4-1BB), CD150 (SLAMFI), CD152 (CTLA4), CD223 (LAG3), CD270 (HVEM), CD272 (BTLA), CD273 (PD-L2), CD274 (PD-L1), CD276 (B7-H3), CD278 (ICOS), CD357 (GITR), DAP10, LAT, NKG2C, SLP76, PD1, LIGHT, TRIM, and ZAP70.

Preferably, the CAR gene further comprises a hinge region (e.g., CD8, CD28, or IgG, and the like). More preferably, the CAR gene further comprises a signal peptide (e.g., CD32). Even more preferably, the CAR gene further comprises an intracellular domain (e.g., IL12, and the like).

In one embodiment of the present invention, the CAR gene comprises a signal peptide, an scFv (comprising the nucleotide sequence from position 70 to position 795 of SEQ ID NO: 25), a hinge region, a transmembrane domain, a costimulatory domain.

In one embodiment of the present invention, the CAR gene comprises and in particular consists of the nucleotide sequence set forth in SEQ ID NO: 25.

In a fifth aspect of the present invention, provided is a vector comprising the CAR gene according to the fourth aspect.

Specifically, the vector is a plasmid, such as a PUC57 plasmid.

Specifically, the CAR gene may be ligated to the vector via a restriction endonuclease (e.g., EcoRI or BamHI) cutting site.

In a sixth aspect of the present invention, provided is a nucleic acid molecule for HPK1, namely a minicircle DNA, which comprises from 5′ to 3′ in sequence: a target sequence (TSF), a left homology arm (LHA), a promoter, a target gene, a polyA, a right homology arm (RHA), a target sequence (TSF).

Specifically, the target sequence comprises the 3′ end of SEQ ID NOs: 1-6 added with a PAM region. The PAM is typically a 2-6 base pair sequence, such as 5′-NGG-3′ (wherein N is any base), 5′-NGA-3′, 5′-YG-3′ (wherein Y is a pyrimidine), 5′TTN-3′, or 5′-YTN-3′. In specific embodiments, the added PAM may be adjusted according to the type of the nuclease.

Specifically, the TSF comprises or consists of the nucleotide sequence set forth in SEQ ID NO: 7 and/or 8, or a nucleotide sequence having at least 70%, 75%, 80%, 85%, 90%, 91%, 92%, 93%, 94%, 95%, 96%, 97%, 98%, or 99% identity or complementarity to the nucleotide sequence set forth in SEQ ID NO: 7 and/or 8, or the sequence of the TSF is set forth in SEQ ID NO: 7 and/or SEQ ID NO: 8.

In a seventh aspect of the present invention, provided is a nucleic acid molecule for HPK1, namely a single-stranded DNA, which is from 5′ to 3′ in sequence:
Sequence A. a gRNA target region, a left homology arm (LHA), a promoter, a target gene, a polyA, a right
homology arm (RHA), a gRNA target region.
Sequence B. a gRNA non-target region, a left homology arm (LHA), a promoter, a target gene, a polyA, a right
homology arm (RHA), a gRNA target region, or a gRNA target region, a left homology arm (LHA), a promoter, a
target gene, a polyA, a right homology arm (RHA), a gRNA non-target region.
Sequence C. The sequence A described above paring to a gRNA non-target region+an overlap region fragment, or
Sequence D. The sequence B described above paring to a gRNA target region+an overlap region fragment or/and an overlap region+a gRNA non-target region fragment.

The single-stranded DNA may comprise 0, 1, 2, 3, 4, 5, 6, 7, 8, 9 or 10 gRNA target region(s) and/or gRNA non-target region(s). The number of bases of the overlap region fragment may be 0-500 bp, e.g., 0, 5, 10, 15, 20, 25, 30, 50, 100, 150, 200, 250, 300, 350, 400, 450, or 500 bp. Preferably, the single-stranded DNA may be subjected to phosphorothioate modification between 1-5 bases (such as 1, 2, 3, 4, or 5 bases) at the 5′ end and/or 3′ end.

Specifically, the gRNA target region comprises the nucleotide sequence set forth in SEQ ID NO: 9 and/or 11, or the sequence of the gRNA target region is set forth in SEQ ID NO: 9 and/or 11. The gRNA non-target region comprises the nucleotide sequence set forth in SEQ ID NO: 10 and/or 12, or the sequence of the gRNA non-target region is set forth in SEQ ID NO: 10 and/or 12. Preferably, the gRNA target region is the sequence set forth in SEQ ID NO: 11, the gRNA non-target region is the sequence set forth in SEQ ID NO: 12.

Specifically, the left homology arm and the right homology arm have a length of 40-2000 bp (e.g., 40, 100, 200, 300, 400, 500, 600, 700, 800, 900, 1000, 1500, or 2000 bp), respectively; in one embodiment of the present invention, the left homology arm and the right homology arm comprise the nucleotide sequence set forth in SEQ ID NOs: 13 and 14, respectively, or are set forth in SEQ ID NOs: 13 and 14, respectively; in another embodiment of the present invention, the left homology arm and the right homology arm comprise the nucleotide sequence set forth in SEQ ID NOs: 15 and 16, respectively, or are set forth in SEQ ID NOs: 15 and 16, respectively; in another embodiment of the present invention, the left homology arm and the right homology arm comprise the nucleotide sequence set forth in SEQ ID NOs: 17 and 18, respectively, or are set forth in SEQ ID NOs: 17 and 18, respectively. Specifically, the promoter may be an EF1α promoter, a CMV promoter, an SFFV promoter, and the like. In one embodiment of the present invention, the promoter is an EF1α promoter, the nucleotide sequence thereof is set forth in SEQ ID NO: 19.

Specifically, the polyA may be BGHpA, SV40 polyA, or WPRE, the nucleotide sequences thereof are set forth in SEQ ID NOs: 20-22, respectively; in one embodiment of the present invention, the polyA is SV40 polyA.

Specifically, a restriction endonuclease (e.g., BamHI or EcoRI) cutting site is also included between the promoter and the polyA.

In an eighth aspect of the present invention, provided is a vector comprising the nucleic acid molecule according to the sixth or seventh aspect.

Specifically, the vector is a plasmid, such as a pMC.BESPX-MCS2 plasmid.

Specifically, the nucleic acid molecule according to the sixth or seventh aspect may be ligated to the vector via a restriction endonuclease (e.g., SpeI or ApaI) cutting site.

In a ninth aspect of the present invention, provided is a homologous recombination repair template DNA, which comprises a target gene and a homology arm.

Specifically, the minicircle DNA nucleic acid molecule for HPK1 comprises from 5′ to 3′ in sequence: a target sequence (TSF), a left homology arm (LHA), a promoter, a target gene, a polyA, a right homology arm (RHA), a target sequence (TSF).

The single-stranded DNA nucleic acid molecule for HPK1 is from 5′ to 3′ in sequence:
Sequence A. a gRNA target region, a left homology arm (LHA), a promoter, a target gene, a polyA, a right homology arm (RHA), a gRNA target region.
Sequence B. a gRNA non-target region, a left homology arm (LHA), a promoter, a target gene, a polyA, a right homology arm (RHA), a gRNA target region.
Sequence C. The sequence A described above paring to a gRNA non-target region+an overlap region fragment: namely, a gRNA target region paring to a gRNA non-target region+an overlap region, a left homology arm (LHA), a promoter, a target gene, a polyA, a right homology arm (RHA), a gRNA target region paring to an overlap region+a gRNA non-target region. or
Sequence D. The sequence B described above paring to a gRNA target region+an overlap region fragment or/and an overlap region+a gRNA non-target region fragment, such as a gRNA non-target region paring to a gRNA target region+an overlap region, a left homology arm (LHA), a promoter, a target gene, a polyA, a right homology arm (RHA), a gRNA target region paring to an overlap region+a gRNA non-target region.

Specifically, the target gene may be a CAR gene, for example as described in the fourth aspect of the present invention.

Specifically, the TSF comprises the nucleotide sequence set forth in SEQ ID NO: 7 and/or 8, or the sequence of the TSF is set forth in SEQ ID NO: 7 and/or SEQ ID NO: 8.

Specifically, the gRNA target region comprises the nucleotide sequence set forth in SEQ ID NO: 9 and/or 11, or the sequence of the gRNA target region is set forth in SEQ ID NO: 9 and/or 11. The gRNA non-target region comprises the nucleotide sequence set forth in SEQ ID NO: 10 and/or 12, or the sequence of the gRNA non-target region is set forth in SEQ ID NO: 10 and/or 12. Preferably, the gRNA target region is the sequence set forth in SEQ ID NO: 11, the gRNA non-target region is the sequence set forth in SEQ ID NO: 12.

Specifically, the left homology arm and the right homology arm have a length of 40-2000 bp (e.g., 40, 100, 200, 300, 400, 500, 600, 700, 800, 900, 1000, 1500, or 2000 bp), respectively; in one embodiment of the present invention, the left homology arm and the right homology arm comprise the nucleotide sequence set forth in SEQ ID NOs: 13 and 14, respectively, or are set forth in SEQ ID NOs: 13 and 14, respectively; in another embodiment of the present invention, the left homology arm and the right homology arm comprise the nucleotide sequence set forth in SEQ ID NOs: 15 and 16, respectively, or are set forth in SEQ ID NOs: 15 and 16, respectively; in another embodiment of the present invention, the left homology arm and the right homology arm comprise the nucleotide sequence set forth in SEQ ID NOs: 17 and 18, respectively, or are set forth in SEQ ID NOs: 17 and 18, respectively. Specifically, the promoter may be an EF1α promoter, a CMV promoter, an SFFV promoter, and the like. In one embodiment of the present invention, the promoter is an EF1α promoter, the nucleotide sequence thereof is set forth in SEQ ID NO: 19.

Specifically, the polyA may be BGHpA, SV40 polyA, or WPRE, the nucleotide sequences thereof are set forth in SEQ ID NOs: 20-22, respectively; in one embodiment of the present invention, the polyA is SV40 polyA.

In a tenth aspect of the present invention, provided is a vector comprising the homologous recombination repair template DNA according to the ninth aspect.

In some embodiments of the present invention, the vector is a vector that can produce an AAV, a minicircle DNA, a double-stranded DNA, or a single-stranded DNA, particularly a plasmid that can produce a minicircle DNA, such as pMC.BESPX-MCS2.

In other embodiments of the present invention, the vector is a vector that can produce an AAV, a minicircle DNA, a double-stranded DNA, or a single-stranded DNA, particularly a single-stranded DNA vector that can be chemically synthesized, or a single-stranded DNA vector that is obtained by cutting a dsDNA with a exonuclease or by other biological or chemical methods.

In other embodiments of the present invention, the vector is an AAV, a minicircle DNA, a double-stranded DNA, or a single-stranded DNA, particularly a minicircle DNA or a single-stranded DNA.

In an eleventh aspect of the present invention, provided is a method for preparing the vector according to the tenth aspect, which comprises the step of sequentially constructing the nucleic acid molecule according to the sixth or seventh aspect of the present invention and the CAR gene according to the fourth aspect into a vector. Specifically, the method for preparing the minicircle DNA vector comprises the following steps:
(1) performing enzyme digestion, ligation, transformation, screening on the nucleic acid molecule according to the sixth aspect of the present invention, a minicircle parent plasmid (such as pMC.BESPX-MCS2);
(2) performing enzyme digestion, ligation, transformation, screening on the vector according to the fifth aspect of the present invention, the plasmid obtained by screening in step (1);
optionally, (3) performing culture under induction on the plasmid extracted in step (2) by using arabinose, and extracting the plasmid after the culture under induction.

Specifically, step (1) and/or (2) further comprise a step of performing sequencing identification after the ligation. Specifically, the transformation described in step (1) and/or (2) is to transform the plasmid obtained after the ligation into a minicircle competent (e.g., ZYCY10P3S2TE.coil) cell.

Specifically, the medium for the culture under induction of step (3) is TB medium, wherein the concentration of the arabinose is 0.01-1%.

Specifically, the method for preparing the single-stranded DNA vector comprises the following steps:
(1) synthesizing a single-stranded DNA primer designed for the homologous recombination repair template DNA according to the ninth aspect of the present invention by a company, wherein one of the primers is subjected to phosphorothioate modification at the 5′ end for 3-5 bases.
(2) using the minicircle DNA vector formed according to the eleventh aspect of the present invention as a template to form a dsDNA by PCR, recovering, cutting with an exonuclease, and purifying.

In a twelfth aspect of the present invention, provided is a gRNA having a target domain complementary to a target sequence at the HPK1 site (e.g., HPK1 EXON).

In some embodiments of the present invention, the target domain sequence comprises or consists of the nucleotide sequence set forth in any one of SEQ ID NOs: 1-6.

In some embodiments of the present invention, the gRNA comprises and in particular consists of the nucleotide sequence set forth in SEQ ID NO: 23 or 24.

Specifically, the gRNA may also comprise a chemical modification of the base, such as methylation modification or thio-modification or a combination of both; more specifically, 1-5 bases (e.g., 1, 2, 3, 4, or 5 bases) at the 5′ end and/or 3′ end of the gRNA are subjected to 2′-O-methylation modification and/or phosphorothioate modification; in some embodiments of the present invention, 3 bases at the 3′ end of the gRNA are subjected to 2′-O-methylation modification.

In a thirteenth aspect of the present invention, provided is a composition comprising the gRNA according to the twelfth aspect, and a CRISPR nuclease.

Specifically, the nuclease may be SpCas9, SaCas9, eSpCas9, Cas12a, Cas13, or cpf1 and mutants thereof; in some embodiments of the present invention, the nuclease may be eSpCas9.

In a fourteenth aspect of the present invention, provided is a complex of the gRNA according to the twelfth aspect and a nuclease (RNP).

Specifically, the nuclease may be SpCas9, SaCas9, eSpCas9, Cas12a, Cas13, or cpf1; in some embodiments of the present invention, the nuclease may be eSpCas9.

In a fifteenth aspect of the present invention, provided is use of the method according to the first or second aspect of the present invention and a site-specific integrated immune cell prepared by the method, the CAR gene according to the fourth aspect and a vector comprising the CAR gene, the nucleic acid molecule according to the sixth or seventh aspect and a vector comprising the nucleic acid molecule, the homologous recombination repair template DNA according to the ninth aspect and a vector comprising the homologous recombination repair template DNA, the gRNA according to the twelfth aspect, the composition according to the thirteenth aspect, or the complex according to the fourteenth aspect in preparing a gene therapy medicine.

Specifically, diseases requiring gene therapy include, for example, malignant tumors, genetic diseases (such as hemophilia, congenital amaurosis, or cystic fibrosis), cardiovascular diseases, infectious diseases (such as AIDS, and the like), autoimmune diseases (such as systemic lupus erythematosus, rheumatoid arthritis, ankylosing spondylitis, or psoriasis, and the like), or immune rejection, and the like, particularly malignant tumors.

Specifically, the malignant tumor may be, for example, lymphoma, chronic lymphocytic leukemia (CLL), B-cell acute lymphocytic leukemia (B-ALL), acute lymphocytic leukemia, acute myelocytic leukemia, non-Hodgkin lymphoma (NHL), diffuse large cell lymphoma (DLCL), multiple myeloma, renal cell carcinoma (RCC), neuroblastoma, colorectal cancer, breast carcinoma, ovarian carcinoma, melanoma, sarcoma, prostate cancer, lung cancer, esophageal cancer, hepatocellular carcinoma, pancreatic cancer, astrocytoma, mesothelioma, head and neck cancer, and medulloblastoma, and the like.

In a sixteenth aspect of the present invention, provided is a method for treating a disease, wherein the treatment method comprises administering to a subject in need a therapeutically effective amount of the method according to the first or second aspect of the present invention and a site-specific integrated immune cell prepared by the method, the CAR gene according to the fourth aspect and a vector comprising the CAR gene, the nucleic acid molecule according to the sixth or seventh aspect and a vector comprising the nucleic acid molecule, the homologous recombination repair template DNA according to the ninth aspect and a vector comprising the homologous recombination repair template DNA, the gRNA according to the twelfth aspect, the composition according to the thirteenth aspect, or the complex according to the fourteenth aspect.

Specifically, the immune cell may be of autologous origin (e.g., from a subject suffering from a disease requiring gene therapy) or of allogeneic origin (e.g., from a healthy donor).

Specifically, the disease includes, for example, malignant tumors, genetic diseases (such as hemophilia, congenital amaurosis, or cystic fibrosis), cardiovascular diseases, infectious diseases (such as AIDS, and the like), autoimmune diseases (such as systemic lupus erythematosus, rheumatoid arthritis, ankylosing spondylitis, or psoriasis, and the like), or immune rejection, and the like, particularly malignant tumors.

Specifically, the malignant tumor may be, for example, lymphoma, chronic lymphocytic leukemia (CLL), B-cell acute lymphocytic leukemia (B-ALL), acute lymphocytic leukemia, acute myelocytic leukemia, non-Hodgkin lymphoma (NHL), diffuse large cell lymphoma (DLCL), multiple myeloma, renal cell carcinoma (RCC), neuroblastoma, colorectal cancer, breast carcinoma, ovarian carcinoma, melanoma, sarcoma, prostate cancer, lung cancer, esophageal cancer, hepatocellular carcinoma, pancreatic cancer, astrocytoma, mesothelioma, head and neck cancer, or medulloblastoma, and the like.

The method provided by the present invention comprises designing a homologous recombination repair template targeting a specific site (such as HPK1), simultaneously introducing the homologous recombination repair template and a gene editing system into an immune cell in a physical or chemical mode, and realizing gene knockout and target gene introduction in one step, which can accurately integrate a target gene (such as CAR gene) into a specific site (such as HPK1) of the immune cell, and prepare a cell with knockout of the gene at the specific site and stably expressing the target gene. The method has the characteristics of simple preparation, low cost, stable expression, and stable function, and does not influence the characteristics of cell genes. The obtained cells have the characteristics of high killing efficiency, strong infiltration capacity, and the like.

“Treating” described herein refers to after the disease has begun to progress, slowing, interrupting, arresting, controlling, stopping, reducing or reversing the progression or severity of a sign, symptom, disorder, condition or disease, but does not necessarily involve the complete elimination of all disease-related signs, symptoms, conditions or disorders.

“Effective amount” described herein refers to an amount or dose of the product described herein which provides the desired treatment after administration to a subject in single or multiple doses.

The “subject” described herein may be a human or non-human mammal or an organ or cell thereof, and the non-human mammal may be a wild animal, a zoo animal, an economic animal, a companion animal, a laboratory animal, and the like. Preferably, the non-human mammal includes, but is not limited to, a pig, a cow, a sheep, a horse, a donkey, a fox, a racoon dog, a mink, a camel, a dog, a cat, a rabbit, a murine (e.g., a rat, a mouse, a guinea pig, a hamster, a gerbil, a chinchilla, a squirrel), or a monkey, or the like.

### BRIEF DESCRIPTION OF THE DRAWINGS

FIG. 1 shows the vector structure, wherein MC is the schematic diagram of the design of the homologous recombination repair template with an enzyme cutting site, and A, B, C, and D are single-stranded DNAs with different structures, respectively.

FIG. 2 shows the plasmid map of the constructed minicircle DNA vector.

FIG. 3 shows the schematic diagram of the purification of the minicircle DNA vector (lane MC), single-stranded DNA A (lane A, corresponding to A in FIG. 1), and single-stranded DNA vector B (lane B, corresponding to B in FIG. 1).

FIG. 4 shows the results of HPK1 knockout efficiency by WB assay, wherein lane 1: control group without electroporation; lane 2: electroporation of RNP alone; lane 3: electroporation of RNP and then lentivirus infection; lane 4: electroporation of minicircle DNA vector and RNP; lane 5: electroporation of single-stranded DNA vector A and RNP; lane 6: electroporation of single-stranded DNA vector B and RNP; lane 7: electroporation of single-stranded DNA vector C and RNP; lane 8: electroporation of single-stranded DNA vector D and RNP.

FIG. 5 shows the results of the CAR positive rate of CD19 by flow cytometry, wherein the first row represents, in order from left to right, control group without electroporation, electroporation of RNP alone, electroporation of RNP and then lentivirus infection, and electroporation of minicircle DNA vector and RNP; the second row represents, in order from left to right, electroporation of single-stranded DNA vector A and RNP, electroporation of single-stranded DNA vector B and RNP, electroporation of single-stranded DNA vector C and RNP, and electroporation of single-stranded DNA vector D and RNP.

FIG. 6 shows the results of the release levels of IFN-γ by ELISA, wherein T cell represents a control group without electroporation, HPK1 KOT cell represents electroporation of RNP alone, HPK1 KO CD19 CART cell (Lenti) represents electroporation of RNP and then lentivirus infection, HPK1 KO CD19 CART cell (MC) represents electroporation of minicircle DNA vector and RNP, HPK1 KO CD19 CART cell (A ssDNA) represents electroporation of single-stranded DNA vector A and RNP, HPK1 KO CD19 CART cell (B ssDNA) represents electroporation of single-stranded DNA vector B and RNP, HPK1 KO CD19 CART cell (C ssDNA) represents electroporation of single-stranded DNA vector C and RNP, and HPK1 KO CD19 CART cell (D ssDNA) represents electroporation of single-stranded DNA vector D and RNP.

FIG. 7A shows the results of the killing efficiency of CART cells against target cells Daudi-luci by Luciferase assay, wherein T cell represents a control group without electroporation, HPK1 KO T cell represents electroporation of RNP alone, HPK1 KO-CD19-CART cell (Lenti) represents electroporation of RNP and then lentivirus infection, HPK1 KO-CD19-CART cell (MC) represents electroporation of minicircle DNA vector and RNP, HPK1 KO-CD19-CART cell (A ssDNA) represents electroporation of single-stranded DNA vector A and RNP, HPK1 KO-CD19-CART cell (B ssDNA) represents electroporation of single-stranded DNA vector B and RNP, HPK1 KO-CD19-CART cell (C ssDNA) represents electroporation of single-stranded DNA vector C and RNP, and HPK1 KO-CD19-CART cell (D ssDNA) represents electroporation of single-stranded DNA vector D and RNP.

FIG. 7B shows the results of the killing efficiency of CART cells against target cells Raji-luci by Luciferase assay, wherein T cell represents a control group without electroporation, HPK1 KO T cell represents electroporation of RNP alone, HPK1 KO-CD19-CART cell (Lenti) represents electroporation of RNP and then lentivirus infection, HPK1 KO-CD19-CART cell (MC) represents electroporation of minicircle DNA vector and RNP, HPK1 KO-CD19-CART cell (A ssDNA) represents electroporation of single-stranded DNA vector A and RNP, HPK1 KO-CD19-CART cell (B ssDNA) represents electroporation of single-stranded DNA vector B and RNP, HPK1 KO-CD19-CART cell (C ssDNA) represents electroporation of single-stranded DNA vector C and RNP, and HPK1 KO-CD19-CART cell (D ssDNA) represents electroporation of single-stranded DNA vector D and RNP.

FIG. 7C shows the results of the killing efficiency of CART cells against target cells K562-luci by Luciferase assay, wherein T cell represents a control group without electroporation, HPK1 KO T cell represents electroporation of RNP alone, HPK1 KO-CD19-CART cell (Lenti) represents electroporation of RNP and then lentivirus infection, HPK1 KO-CD19-CART cell (MC) represents electroporation of minicircle DNA vector and RNP, HPK1 KO-CD19-CART cell (A ssDNA) represents electroporation of single-stranded DNA vector A and RNP, HPK1 KO-CD19-CART cell (B ssDNA) represents electroporation of single-stranded DNA vector B and RNP, HPK1 KO-CD19-CART cell (C ssDNA) represents electroporation of single-stranded DNA vector C and RNP, and HPK1 KO-CD19-CART cell (D ssDNA) represents electroporation of single-stranded DNA vector D and RNP.

### DETAILED DESCRIPTION

Unless otherwise defined, all scientific and technical terms used in the present invention have the same meaning as commonly understood by those skilled in the art to which the present invention relates.

The disclosures of the various publications, patents, and published patent specifications cited herein are hereby incorporated by reference in their entireties.

Technical solutions in the embodiments of the present invention will be described clearly and completely below with reference to the drawings. It is apparent that the described embodiments are only a part of the embodiments of the present invention, but not all of them. Based on the embodiments of the present invention, all other embodiments derived by those of ordinary skill in the art without creative work shall fall within the protection scope of the present invention.

Examples without specified techniques or conditions are all implemented according to techniques or conditions described in the literature in the art or according to product instructions. Reagents or instruments without specified manufacturers used herein are conventional products obtained through formal procurement channels.

### Example 1: Preparation of Minicircle DNA Vector Comprising Target Gene CAR

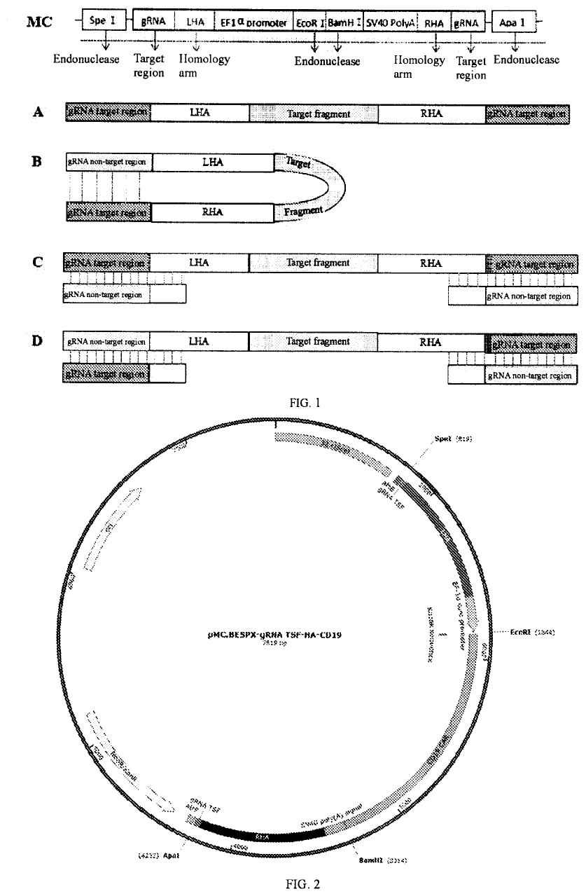

1. The scFv sequence of the CD 19 CAR was used for sequentially splicing a signal peptide, an scFv, a hinge region, a transmembrane domain, and a costimulatory domain sequence, and EcoRI and BamHI enzyme cutting sites were designed at the left end and the right end of the sequence. The whole-gene synthesis (BGI) of the CAR sequence of the target gene (SEQ ID NO: 25) was carried out according to the structure described above, which was named as PUC57-CD19 CAR.
2. The design and synthesis of the HPK1 site homologous recombination repair template, wherein the method was as follows: A gRNA sequence was designed on the EXON of HPK1 genome, an HPK1 gRNA target region (the target sequence is SEQ ID NO: 7), a left homology arm (SEQ ID NO: 13), an EF1 α promoter (SEQ ID NO: 19), an SV40 polyA (SEQ ID NO:21), a right homology arm (SEQ ID NO:14), and a gRNA target region (the target sequence is SEQ ID NO: 7) were spliced in sequence, and SpeI and ApaI enzyme cutting sites (shown in MC in FIG. 1) were designed at the left end and the right end of the sequence. The whole-gene synthesis (BGI) of the HPK1 site homologous recombination sequence was carried out according to the structure described above, which was named as PUC57-HPK1 HA.
3. The construction of the minicircle DNA vector comprising the target gene CAR, wherein the method was as follows:
(1) plasmid extraction was performed by using a plasmid extraction kit (TIANGEN) to extract the PUC57-CD19 CAR and the PUC57-HPK1 HA described above and the minicircle parent pMC.BESPX-MCS2 provided by the minicircle DNA vector kit.
(2) SpeI & ApaI enzyme digestion was performed on PUC57-HPK1 HA and pMC.BESPX-MCS2 and detected by gel electrophoresis, and the target vector and fragment were respectively recovered by using a DNA gel recovery kit (TIANGEN) to give an enzyme digestion product.
(3) ligation reaction; the vector and the fragment recovered by the enzyme digestion described above were ligated, transformed into minicircle competence (ZYCY10P3S2TE.coil), coated on a Kan resistant plate, and cultured overnight.
(4) cloning and identification: monoclonal bacteria were selected, and the plasmids were extracted by a plasmid mini kit (TIANGEN), identified as correct by SpeI & ApaI double enzyme digestion electrophoresis, the sequencing was performed, wherein the sequencing results showed that: the gene sequence was correct, and the pMC.BESPX-HPK1 HA plasmid was successfully constructed.
4. The target CAR gene was constructed into the pMC.BESPX-HPK1 HA plasmid according to the process described above to construct a pMC.BESPX-HPK1 HA-CD19 CAR plasmid. Identification by sequencing showed that the construction was successful. The plasmid bacterial solution, which was successfully constructed, was preserved with −80° C.glycerol for subsequent use. The plasmid map information is shown in FIG. 2.
5. Expression induction and extraction of pMC.BESPX-HPK1 HA-CD19 CAR plasmid
(1) The successfully constructed minicircle DNA vector glycerol bacteria were inoculated into a kan resistant LB medium and cultured with shaking at 30° C.′ until the OD₆₀₀ was approximately equal to 0.6.
(2) The bacteria was inoculated into a TB medium and cultured with shaking overnight.
(3) The pH and OD₆₀₀ of a small amount of the bacterial solution was measured the next day to ensure that pH was between 6.5 and 7.5 and the OD₆₀₀ was between 6 and 8. A TB medium containing 0.1% L-arabinose was added for culture under induction for 4 h. After the induction was completed, a small amount of the induced bacterial solution was extracted in a small amount, and the quality of the pMC.BESPX-HPK1 HA-CD19 CAR plasmid was detected by EcoRI & BamHI enzyme digestion electrophoresis.
(4) After the quality was qualified, the MC.BESPX-HPK1 HA-CD19 CAR plasmid was extracted in a large amount using a QIAGEN EndoFree Plasmid Maxi Kit according to the operation steps, the concentration was measured, and after the quality was qualified, the plasmid was stored in a freezer at −20° C.′ for later use.

### Example 2: Preparation of Single-Stranded DNA Vector Comprising Target Gene CAR

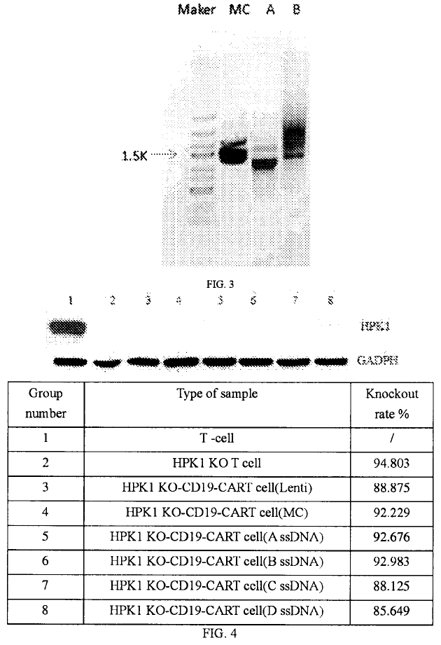

Primers of the single-stranded DNA (the schematic diagram is shown in A-D in FIG. 1) were designed using the MC.BESPX-HPK1 HA-CD19 CAR as a template, and the primers were synthesized by Tsingke Biotechnology Co., Ltd. Then PrimeSTAR® HS DNA Polymerase (TAKARA) PCR was performed to give a dsDNA fragment. Then a single-stranded DNA was formed after the T7 Exonuclease (NEB) digestion, which was purified and recovered by a NucleoSpin Gel and PCR Clean-up Kit (Macherey-Nage), the concentration was measured, and after the quality was qualified, the single-stranded DNA was stored in a freezer at −20° C. for later use. C and D are respectively obtained by annealing and extending A and B and the overlap region pairing thereto. A schematic diagram of single-stranded DNA purification is shown in FIG. 3. The single-stranded DNA A sequentially is a gRNA target region (SEQ ID NO: 9), a left homology arm (SEQ ID NO: 13), an EF1 a promoter (SEQ ID NO: 19), a target gene (SEQ ID NO: 25), an SV40 polyA (SEQ ID NO: 21), a right homology arm (SEQ ID NO: 14), and a gRNA target region (SEQ ID NO: 9); the single-stranded DNA B sequentially is a gRNA non-target region (SEQ ID NO: 10), a left homology arm (SEQ ID NO: 13), an EF1 a promoter (SEQ ID NO: 19), a target gene (SEQ ID NO: 25), an SV40 polyA (SEQ ID NO: 21), a right homology arm (SEQ ID NO: 14), and a gRNA target region (SEQ ID NO: 9).

The sequences of the synthesized primers are shown in Table 1.

TABLE 1

| Name | |
|---|---|
| of | |
| primer | Primer sequence |
| A-Fw | T*A*A*T*A*ccatcttcagtgccaccaggtcccctgcgcagt |
| | gcagaggag (SEQ ID NO: 26) |
| A-Rv | CAACTGAGAGAACTCAAAGGTTACCCCAG (SEQ ID NO: |
| | 27) |
| B-Fw | T*A*A*T*A*ggacctggtg gcactgaaga |
| | tggcctgcgcagtgcagaggag (SEQ ID NO: 28) |
| B-Rv | taataggacctggtg gcactgaaga |
| | tggggtcagaaatgggaatttta (SEQ ID NO: 29) |

Wherein, * represents a phosphorothioated modification.

### Example 3: Preparation of Cas9 and gRNA

The sequence information of the HPK1 gRNA (SEQ ID NO: 24) was designed and obtained according to the website https://www.genscript.com/gma-design-tool.htm1, synthesized by Genscript bio corporation, and modified by methylation on three bases at the 3′ end of the gRNA, and the eSpCas9 protein was purchased from Genscript bio corporation (Cat. No.: Z03622).

### Example 4: Preparation of HPK1 Site-Specific Integrated CART Cells Based on Homologous Recombination Repair Template

### 1. PBMC Isolation

Peripheral blood samples PBMCs of volunteers were collected for isolation. The blood samples were diluted with PBS buffer at equal volumes and gently added to a Ficoll lymphocyte isolation solution. The mixture was centrifuged at 800 g for 20 min. After centrifugation, the middle annular milk white lymphocytes were pipetted, transferred into a new 50 mL centrifuge tube, washed twice with PBS, counted, resuspended in a cryopreservation solution, and aliquoted at 1 mL each vial for cryopreservation in liquid nitrogen.

### 2. T Cell Thawing, Activation, and Culture

The cryopreserved PBMCs of the healthy volunteers were thawed in a water bath at 37° C., washed twice in a 1640 medium and counted. The cells were resuspended. Activation and culture were carried out with the addition of CD3/CD28 beads for 48 h.

3. Preparation of HPK1 KO CD19 CART Cells by T Cells with Electroporation of a Homologous Recombination Repair Template
(1) Cell treatment: T cells which were activated and cultured for 48 h were collected in a 15 mL centrifuge tube, and washed once with PBS. Magnetic beads were removed on a magnetic rack. The cells were counted, the cell density was adjusted to 1.0×10⁷ cells/mL using electroporation buffer, and electroporation was carried out.
(2) Electroporation system preparation: Cas9 protein and the gRNA (prepared in Example 3) were mixed well and incubated at room temperature for 10 min, the homologous recombination repair template for the desired electroporation was added, the mixture was mixed well with 100 μL of cell suspension, and electroporation was carried out.
(3) Electroporation: Electroporation was carried out using the Invitrogen Neon (Thermo Fisher) electrotransfection system according to the parameters 1300 V, 20 ms, and 1 pulse, and the cell suspension was transferred to a medium, and left to stand and incubated in an incubator at 37° C. with 5% CO₂.
   4. Preparation of HPK1 KO T Cells by T Cells with Electroporation of Cas9/gRNA Ribonucleoprotein Complex (CRISPR-Cas9RNP, Hereinafter Abbreviated as RNP)
(1) Cell treatment: T cells which were activated and cultured for 48 h were collected in a 15 mL centrifuge tube, and washed once with PBS. Magnetic beads were removed on a magnetic rack. The cells were counted. The cell density was adjusted to 1.0×10⁷ cells/mL using electroporation buffer, and electroporation was carried out.
(2) Electroporation system preparation: Cas9 protein and the gRNA (prepared in Example 3) were mixed well, incubated at room temperature for 10 min, and mixed well with 100 μL of cell suspension, and electroporation was carried out.
(3) Electroporation: Electroporation was carried out using the Invitrogen Neon (Thermo Fisher) electrotransfection system according to the parameters 1300 V, 20 ms, and 1 pulse, and the cell suspension was transferred to a preheated medium, and left to stand and incubated in an incubator at 37° C. with 5% CO₂.
   5. Preparation of HPK1 KO CD19 CART Cells (Lenti) by Lentivirus Infection and T Cells with Electroporation of RNP
(1) Lentivirus infection of T cells: Infection was carried out by adding a lentivirus to T cells which were activated and cultured for 48 h, and after the infection was carried out for 24 h, electroporation was carried out.
(2) Electroporation cell treatment: CART cells which were infected for 24 h were collected in a 15 mL centrifuge tube, and washed once with PBS. Magnetic beads were removed on a magnetic rack. The cells were counted. The cell density was adjusted to 1.0×10⁷ cells/mL using electroporation buffer, and electroporation was carried out.
(3) Electroporation system preparation: Cas9 protein and the gRNA (prepared in Example 3) were mixed well, incubated at room temperature for 10 min, and mixed well with 100 μL of cell suspension, and electroporation was carried out.
(4) Electroporation: Electroporation was carried out using the Invitrogen Neon (Thermo Fisher) electrotransfection system according to the parameters 1300 V, 20 ms, and 1 pulse, and the cell suspension was transferred to a medium, and left to stand and incubated in an incubator at 37° C.′ with 5% CO₂.

### 6. HPK1 Knockout Efficiency of Prepared CART Cells Targeting CD19 Detected by Western Bloting

(1) Total protein extraction and concentration measurement: T cells, T cells with knockout of HPK1 (HPKIKO T cells), and CART cells (HPKIKO CD19 CART cells) were collected and washed twice with 1 mL of PBS. 20 μL of RIPA was added to the sample of each group, and the cells were lysed at 4° C. for 1 h and centrifuged at 12000 g at 4° C. for 15 min. The lysate supernatant was collected and the total protein concentration was measured by BCA.
(2) Electrophoresis: 20 μg of protein was loaded and subjected to SDS-PAGE electrophoresis.
(3) Membrane transfer: The protein was transferred to a PVDF membrane at 300 mA for 40 min.
(4) Blocking: The membrane was blocked with 5% skim milk for 2 h.
(5) Incubation of primary antibody: Antibodies were prepared. The HPK1 primary antibody and the GAPDH primary antibody were diluted in a 1:1000 ratio, and the mixture was incubated at 4° C. overnight.
(6) Membrane washing: The primary antibody incubation solution was discarded, and the membrane was washed 3 times with a TBST solution, 5 min each time.
(7) Incubation of secondary antibody: The secondary antibody was diluted in a 1:5000 ratio, and incubated at room temperature in the dark for 45 min.
(8) Membrane washing: The membrane was washed 4 times with TBST, 10 min each time.
(9) Color development: Color was developed using an ECL luminescence kit and photographed, and the data were processed.

The results are shown in FIG. 4. The HPK1 knockout efficiency of cells with simultaneous electroporation of the minicircle DNA vector and RNP was about 92% (lane 4), and the HPK1 knockout efficiency of cells with simultaneous electroporation of the single-stranded DNA A and RNP (lane 5) and cells with simultaneous electroporation of the single-stranded DNA B and RNP (lane 6) was about 92%, which was not greatly different from that of cells with electroporation of RNP alone (lane 2).

### 7. Flow Cytometry for Efficiency of Knock-In of HPK1 Gene to CAR Gene Targeting CD19

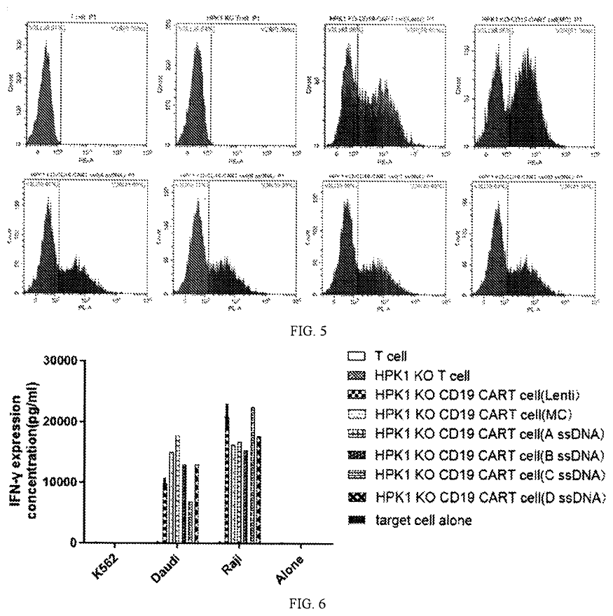

Each group was sampled 2.0×10⁶ cells. The cells were washed twice with 1 mL of 2% BSA/PBS, and aliquoted at 50 μL in 1.5 mL EP tubes, which were NC, the secondary antibody single staining control group, and the experimental group, respectively. 5 μL of Biotinylated-CD19 protein was added to the tube of the experimental group, incubated at 4° C.′ for 1 h in the dark and washed twice with 1 mL of 2% BSA/PBS. The cells were resuspended in 50 μL of 2% BSA/PBS, the PE-Streptavidin secondary antibody was separately added to the secondary antibody single staining control group and the experimental group, and the mixture was incubated at 4° C. for 30 min in the dark. After incubation, the mixture was washed twice with 1 mL of 2% BSA/PBS, the cells were resuspended in 200 μL of 2% BSA/PBS, and the percentage of CD19+ cells was assayed on a machine. The results are shown in FIG. 5. It can be seen that the knock-in efficiency of CAR gene introduction by the minicircle DNA (MC) vector was about 60%, and the knock-in efficiency of CAR gene introduction by the single-stranded DNA (ssDNA) vector was about 40%.

8. Cytokine Release Experiment after In Vitro Co-Incubation of Prepared CART Cells Targeting CD19 and Tumor Cells

Firstly, resting T cells, T cells with knockout of HPK1 (HPK1 KO T cells), CART cells (HPK1 KO CD19 CART cells), and target cells K562, Daudi, and Raji were collected in a 15 mL centrifuge tube, washed twice with a X-VIVO-15 medium without addition, the supernatant was discarded, and the cells were resuspended with a X-VIVO-15 medium without addition, and adjusted to a certain density. The effector cells were mixed with the target cells at a ratio of 1:1. Three replicate wells were made for each sample. The cells were incubated in a X-VIVO-15 medium without addition at 37° C. for 16 h and then centrifuged at 300 g for 5 min. The supernatant was collected for IFN-γ release level detection (IFN-γ ELISA KIT: purchased from Sino Biological Inc., Cat No.: KIT 11725A). The results are shown in FIG. 6: Meanwhile, the IFN-γ release level of the HPK1 KO CD19 CART cell group was higher than that of the T cell group and the HPK1 KO T cell group, and an obvious killing tendency was realized, which was not greatly different from that of the lentivirus infection group (HPK1 KO CD19 CART cell (Lenti)), further proving the success of the CD19 CAR knock-in.

### 9. Killing Experiment of Prepared CART Cells Targeting CD19 on Tumor Cells In Vitro

T cells, T cells with knockout of HPK1 (HPK1 KO T cells), CART cells (HPK1 KO CD19 CART cells), and target cells K562-Luci, Raji-Luci, and Daudi-Luci were collected in a 15 mL centrifuge tube, washed twice with a X-VIVO-15 medium without addition, the supernatant was discarded, and the cells were resuspended with a X-VIVO-15 medium without addition, and adjusted to a certain density. The effector cells were mixed with the target cells at ratios of 0.5:1, 1:1, 2:1, and 3:1. Three replicate wells were made for each sample. After the cells were incubated in a X-VIVO-15 medium without addition at 37° C. for 4 h, 100 μL of 300 μg/mL D-Luciferin potassium salt substrate was added, and luminescence was detected using a microplate reader.

Cell killing efficiency=(1−fluorescence value of target cells and effector cells co-cultured/fluorescence value of target cells cultured alone)×100%

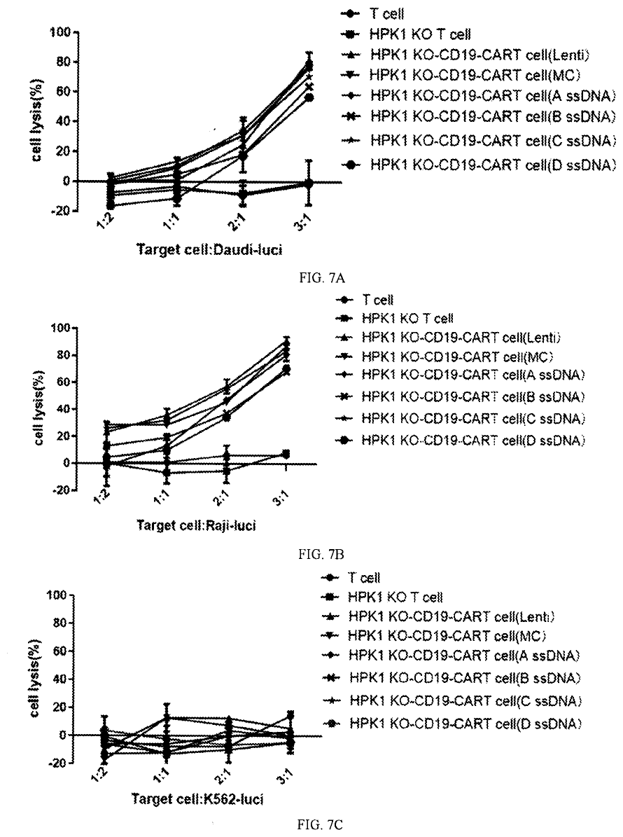

The results are shown in FIG. 7: The cell killing of the HPK1 KO CD19 CART cell group was higher than that of the T cell group and the HPK1 KO T cell group, and an obvious killing tendency was realized, which was not greatly different from that of the lentivirus infection group (HPK1 KO CD19 CART cell (Lenti)), further proving the success of the CD19 CAR knock-in.

The above description is only for the purpose of illustrating the preferred examples of the present invention, and is not intended to limit the scope of the present invention. Any modifications, equivalents, and the like made without departing from the spirit and principle of the present invention shall fall in the protection scope of the present invention.

The foregoing examples and methods described herein may vary based on the abilities, experience, and preferences of those skilled in the art.

The certain order in which the steps of the method are listed in the present invention does not constitute any limitation on the order of the steps of the method.

1. A method for site-specific integration of a target gene into a specific site of an immune cell, comprising the following steps:
S1, constructing a vector comprising a homologous recombination repair template;
S2, introducing a gene editing system and the vector obtained in step S1 into an immune cell together;
optionally, S3, culturing and identifying the immune cell obtained in step S2.

2. The method according to claim 1, wherein the specific site may be genes such as HPK1, PD-1, or TRAC, and the like;
preferably, the specific site is HPK1, particularly HPK1 EXON.

3. The method according to claim 1, wherein the vector in step S1 is selected from: an adeno-associated virus, a minicircle DNA, a double-stranded DNA, or a single-stranded DNA;
preferably, the vector is a minicircle DNA or a single-stranded DNA.

4. The method according to claim 1, wherein the homologous recombination repair template in step S1 comprises1): the minicircle DNA is from 5′ to 3′ in sequence: a target sequence (TSF), a left homology arm (LHA), a promoter, a target gene, a polyA, a right homology arm (RHA), a target sequence (TSF);
or 2):
the single-stranded DNA is from 5′ to 3′ in sequence:
sequence A. a gRNA target region, a left homology arm (LHA), a promoter, a target gene, a polyA, a right homology arm (RHA), a gRNA target region;
sequence B. a gRNA non-target region, a left homology arm (LHA), a promoter, a target gene, a polyA, a right homology arm (RHA), a gRNA target region, or a gRNA target region, a left homology arm (LHA), a promoter, a target gene, a polyA, a right homology arm (RHA), a gRNA non-target region;
sequence C. the sequence A described above paring to a gRNA non-target region+an overlap region fragment; or
sequence D. the sequence B described above paring to a gRNA target region+an overlap region fragment or/and an overlap region+a gRNA non-target region fragment;
wherein the single-stranded DNA may comprise 0, 1, 2, 3, 4, 5, 6, 7, 8, 9 or 10 gRNA target region(s) and gRNA non-target region(s); the number of bases of the overlap region fragment may be 0-500 bp;
preferably, the single-stranded DNA may be subjected to phosphorothioate modification between 1-5 bases at the 5′ end and/or 3′ end.

5. The method according to claim 1, wherein the target gene is a CAR gene;
preferably, the target recognized by the CAR is selected from: ROR1, Her2, L1-CAM, CD19, CD20, CD22, CEA, hepatitis B surface antigen, folate receptor antibody, CD23, CD24, CD30, CD33, CD38, CD276, CD44, EGFR, EGP-2, EGP-4, EPHa2, ErbB2, ErbB3, ErbB4, FBP, fetal acetylcholine receptor, GD2, GD3, HMW-MAA, IL-22R-α, IL-13R-α2, kdr, k light chain, Lewis Y, L1 cell adhesion molecule (CD171), MAGE-A1, mesothelin, MUC1, MUC16, PSCA, NKG2D ligand, NY-ESO-1, MART-1, gp100, oncofetal antigen, TAG72, VEGF-R2, carcinoembryonic antigen (CEA), prostate specific antigen, PSMA, estrogen receptor, progesterone receptor, ephrinB2, CD123, CS-1, c-Met, GD-2, MAGEA3, CE7, Wilms tumor 1 (WT-1), cyclin A1 (CCNA1), BCMA, and interleukin 12, or a combination thereof;
more preferably, the CAR gene comprises the nucleotide sequence set forth in SEQ ID NO: 25.

6. The method according to claim 4, wherein the TSF comprises or consists of the nucleotide sequence set forth in SEQ ID NO: 7 and/or 8, or a nucleotide sequence having at least 70%, 75%, 80%, 85%, 90%, 91%, 92%, 93%, 94%, 95%, 96%, 97%, 98%, or 99% identity or complementarity to the nucleotide sequence set forth in SEQ ID NO: 7 and/or 8.

7. The method according to claim 4, wherein the gRNA target region comprises the nucleotide sequence set forth in SEQ ID NO: 9 and/or 11, or a nucleotide sequence having at least 50%, 55%, 60%, 65%, 70%, 75%, 80%, 85%, 90%, 91%, 92%, 93%, 94%, 95%, 96%, 97%, 98%, or 99% identity to the nucleotide sequence set forth in SEQ ID NO: 9 and/or 11;
preferably, the gRNA target region is the sequence set forth in SEQ ID NO: 9 or/and 11.

8. The method according to claim 4, wherein the gRNA non-target region comprises the nucleotide sequence set forth in SEQ ID NO: 10 and/or 12, or a nucleotide sequence having at least 50%, 55%, 60%, 65%, 70%, 75%, 80%, 85%, 90%, 91%, 92%, 93%, 94%, 95%, 96%, 97%, 98%, or 99% identity to the nucleotide sequence set forth in SEQ ID NO: 10 and/or 12;
preferably, the gRNA non-target region is the sequence set forth in SEQ ID NO: 10 or/and 12.

9. The method according to claim 4, wherein the left homology arm and the right homology arm have a length of 40-2000 bp, respectively;
preferably, the left homology arm and the right homology arm comprise the nucleotide sequence set forth in SEQ ID NOs: 13 and 14, respectively, or are set forth in SEQ ID NOs: 13 and 14, respectively; or
the left homology arm and the right homology arm comprise the nucleotide sequence set forth in SEQ ID NOs: 15 and 16, respectively, or are set forth in SEQ ID NOs: 15 and 16, respectively; or
the left homology arm and the right homology arm comprise the nucleotide sequence set forth in SEQ ID NOs: 17 and 18, respectively, or are set forth in SEQ ID NOs: 17 and 18, respectively.

10. The method according to claim 4, wherein the promoter is an EF1α promoter, a CMV promoter, an SFFV promoter, and the like; preferably, the promoter is an EF1α promoter, the nucleotide sequence thereof is set forth in SEQ ID NO: 19.

11. The method according to claim 4, wherein the polyA is BGHpA, SV40 polyA, or WPRE, the nucleotide sequences thereof are set forth in SEQ ID NOs: 20-22; preferably, the polyA is SV40 polyA.

12. The method according to claim 1, wherein the gene editing system is selected from: CRISPR, ZFN, or TALEN; preferably, the gene editing system is a CRISPR system.

13. The method according to claim 12, wherein a nuclease of the CRISPR system is selected from: SpCas9, SaCas9, eSpCas9, Cas12a, Cas13, and cpf1 and mutants thereof; preferably, the nuclease is eSpCas9.

14. The method according to claim 12, wherein a gRNA of the CRISPR system has a target domain complementary to a target sequence, the target domain sequence comprises the nucleotide sequence set forth in any one of SEQ ID NOs: 1-6;
preferably, the gRNA comprises or consists of the nucleotide sequence set forth in SEQ ID NO: 23 or 24, or a nucleotide sequence having at least 70%, 75%, 80%, 85%, 90%, 91%, 92%, 93%, 94%, 95%, 96%, 97%, 98%, or 99% identity to the nucleotide sequence set forth in SEQ ID NO: 23 or 24.

15. The method according to claim 14, wherein the gRNA may also comprise a chemical modification of the base;
preferably, the chemical modification is methylation modification or thio-modification or a combination of both;
more preferably, 1-5 bases at the 5′ end and/or 3′ end of the gRNA are subjected to 2′-O-methylation modification and/or phosphorothioate modification.

16. The method according to claim 1, wherein the gene editing system in step S2 is a complex of a gRNA and a nuclease.

17. The method according to claim 1, wherein the method for introducing the immune cell in step S2 is selected from: transformation, transfection, heat shock, electroporation, transduction, microinjection, and the like;
preferably, the method for introducing the cell in step S2 is electroporation.

18. The method according to claim 1, wherein the immune cell is selected from: a T cell, an NK cell, a B cell, a macrophage, a dendritic cell, or a monocyte;
preferably, the immune cell is a T cell.

19. The method according to claim 1, wherein the immune cell is of autologous origin or of allogeneic origin.

20. An immune cell prepared by the method according to claim 1.

21. Use of the method according to claim 1 or the immune cell in preparing a gene therapy medicine.

22. The use according to claim 21, wherein diseases requiring gene therapy are selected from: malignant tumors, genetic diseases, cardiovascular diseases, infectious diseases, autoimmune diseases, or immune rejection;
preferably, the diseases are malignant tumors;
more preferably, the malignant tumor is selected from: lymphoma, chronic lymphocytic leukemia, B-cell acute lymphocytic leukemia, acute lymphocytic leukemia, acute myelocytic leukemia, non-Hodgkin lymphoma, diffuse large cell lymphoma, multiple myeloma, renal cell carcinoma, neuroblastoma, colorectal cancer, breast carcinoma, ovarian carcinoma, melanoma, sarcoma, prostate cancer, lung cancer, esophageal cancer, hepatocellular carcinoma, pancreatic cancer, astrocytoma, mesothelioma, head and neck cancer, and medulloblastoma.

23. A method for treating a disease, wherein the treatment method comprises administering to a subject in need thereof a therapeutically effective amount of the immune cell according to claim 20.


SEQUENCE LISTING (29 sequences)

SEQ ID NO: 1 - DNA, 19 bp, synthetic construct
  source 1..19: mol_type = other DNA
gacctggtggcactgaaga

SEQ ID NO: 2 - DNA, 19 bp, synthetic construct
  source 1..19: mol_type = other DNA
gctcgagacaaggtgtcag

SEQ ID NO: 3 - DNA, 20 bp, synthetic construct
  source 1..20: mol_type = other DNA
ggacctggtggcactgaaga

SEQ ID NO: 4 - DNA, 20 bp, synthetic construct
  source 1..20: mol_type = other DNA
accactatgacctgctacag

SEQ ID NO: 5 - DNA, 20 bp, synthetic construct
  source 1..20: mol_type = other DNA
gacctgctacagcggctggg

SEQ ID NO: 6 - DNA, 20 bp, synthetic construct
  source 1..20: mol_type = other DNA
ttgccggcacgccaacatcg

SEQ ID NO: 7 - DNA, 23 bp, synthetic construct
  source 1..23: mol_type = other DNA
ggacctggtggcactgaagatgg

SEQ ID NO: 8 - DNA, 23 bp, synthetic construct
  source 1..23: mol_type = other DNA
ttgccggcacgccaacatcgtgg

SEQ ID NO: 9 - DNA, 23 bp, synthetic construct
  source 1..23: mol_type = other DNA
ccatcttcagtgccaccaggtcc

SEQ ID NO: 10 - DNA, 23 bp, synthetic construct
  source 1..23: mol_type = other DNA
ggacctggtggcactgaagatgg

SEQ ID NO: 11 - DNA, 23 bp, synthetic construct
  source 1..23: mol_type = other DNA
ccacgatgttggcgtgccggcaa

SEQ ID NO: 12 - DNA, 23 bp, synthetic construct
  source 1..23: mol_type = other DNA
ttgccggcacgccaacatcgtgg

SEQ ID NO: 13 - DNA, 800 bp, synthetic construct
  source 1..800: mol_type = other DNA
cctgcgcagtgcagaggaggtaacccaagtgcatgtatgtgacaggtgaacaggcatgtacactgcatagccatccctttgcagacctgccgccttcgggttgtggccaaaccttcaagcacccagcagaggtttagacactacctaggagacgggggcgggggttctaaggcacccccctccaacccagttacgattaagatcatttgcatcacatttacatccagccctgggccttggaggttgagggtggtcccttgggtcccaagacaagctgcccctcagaggctctcagagcccaggggacgggggggtggagcccgttgcacagtcgtgcagtgcagctggggcaggaagcgagagtgaggaggggggaggccacagcccgcggaggcaaggcgggtgcagggcttctggggacggagggaggtgccagaagttgagccctgaggccctgctggcccctgggcgcaggcccagctcaggcccccagggatggacgtcgtggaccctgacattttcaatagagacccccgggaccactatgacctgctacagcggctgggtggcggcacgtatggggaagtctttaaggtgaggaagcccctctgtccccaacaccctacaaccagtgacctttaaggcaagccctcggcagggaggaggctgcaagagggggtcccagttcttgaccttgactcagggatgctgtggagggactctggcaaatggagcctgcctgactgccaacttcccctttgcaaggctcgagacaaggtgtcaggggacctggtggcact

SEQ ID NO: 14 - DNA, 800 bp, synthetic construct
  source 1..800: mol_type = other DNA
gaagatggtgaagatggagcctggtgaggagggaacatggagctccttccccacatccccaccttcgcctcctgggacactgggatcacctcccctgtagctctctccccatgcgagctgccttttctctcccgccctcctcacagatgatgatgtctccacccttcagaaggaaatcctcatattgaaaacttgccggcacgccaacatcgtggcctaccatgggagttatctctggtgatgaacctcagacccaccctggcacccagcccagcccctaacccggagtccccatttcttgatgggggtaccctcagttctttttttttcttttctttttcttttttgagatggagtcttgttctgttgccaggctggagtgcagtggcatgatctcagctcactgcaacctctgcctcctgggttcaagcaattctctgcctcagactcccgagtagctgggattacaggcgctcaccaccctgcccggcaaatttttgtatttttagtagagatggggtttcaccatcttggccaggctggtcttgaactcctgaccttgtgatccactcgcctcggcctccgaaagtgctgggattacaggtgtgatccaccgcgcctggtcttggcaccctcagttctaaccttgaacttccatttctcaccataacccgacctcccatcctggacccccatttttgactgggctaaccctaccatgatcctcactcctaagtcccacttttctgaccagagcagcctctaatctaatctccaatctaaaattcccatttctgacc

SEQ ID NO: 15 - DNA, 400 bp, synthetic construct
  source 1..400: mol_type = other DNA
gggtgcagggcttctggggacggagggaggtgccagaagttgagccctgaggccctgctggcccctgggcgcaggcccagctcaggcccccagggatggacgtcgtggaccctgacattttcaatagagacccccgggaccactatgacctgctacagcggctgggtggcggcacgtatggggaagtctttaaggtgaggaagcccctctgtccccaacaccctacaaccagtgacctttaaggcaagccctcggcagggaggaggctgcaagagggggtcccagttcttgaccttgactcagggatgctgtggagggactctggcaaatggagcctgcctgactgccaacttcccctttgcaaggctcgagacaaggtgtcaggggacctggtggcact

SEQ ID NO: 16 - DNA, 400 bp, synthetic construct
  source 1..400: mol_type = other DNA
gaagatggtgaagatggagcctggtgaggagggaacatggagctccttccccacatccccaccttcgcctcctgggacactgggatcacctcccctgtagctctctccccatgcgagctgccttttctctcccgccctcctcacagatgatgatgtctccacccttcagaaggaaatcctcatattgaaaacttgccggcacgccaacatcgtggcctaccatgggagttatctctggtgatgaacctcagacccaccctggcacccagcccagcccctaacccggagtccccatttcttgatgggggtaccctcagttctttttttttcttttctttttcttttttgagatggagtcttgttctgttgccaggctggagtgcagtggcatgatctcagc

SEQ ID NO: 17 - DNA, 200 bp, synthetic construct
  source 1..200: mol_type = other DNA
aagcccctctgtccccaacaccctacaaccagtgacctttaaggcaagccctcggcagggaggaggctgcaagagggggtcccagttcttgaccttgactcagggatgctgtggagggactctggcaaatggagcctgcctgactgccaacttcccctttgcaaggctcgagacaaggtgtcaggggacctggtggcact

SEQ ID NO: 18 - DNA, 200 bp, synthetic construct
  source 1..200: mol_type = other DNA
gaagatggtgaagatggagcctggtgaggagggaacatggagctccttccccacatccccaccttcgcctcctgggacactgggatcacctcccctgtagctctctccccatgcgagctgccttttctctcccgccctcctcacagatgatgatgtctccacccttcagaaggaaatcctcatattgaaaacttgccggc

SEQ ID NO: 19 - DNA, 212 bp, synthetic construct
  source 1..212: mol_type = other DNA
gggcagagcgcacatcgcccacagtcccgaagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacag

SEQ ID NO: 20 - DNA, 66 bp, synthetic construct
  source 1..66: mol_type = other DNA
ggaagcggagctactaacttcagcctgctgaagcaggctggagacgtggaggagaaccctggacct

SEQ ID NO: 21 - DNA, 122 bp, synthetic construct
  source 1..122: mol_type = other DNA
aacttgtttattgcagcttataatggttacaaataaagcaatagcatcacaaatttcacaaataaagcatttttttcactgcattctagttgtggtttgtccaaactcatcaatgtatctta

SEQ ID NO: 22 - DNA, 591 bp, synthetic construct
  source 1..591: mol_type = other DNA
aatcaacctctggattacaaaatttgtgaaagattgactggtattcttaactatgttgctccttttacgctatgtggatacgctgctttaatgcctttgtatcatgctattgcttcccgtatggctttcattttctcctccttgtataaatcctggttgctgtctctttatgaggagttgtggcccgttgtcaggcaacgtggcgtggtgtgcactgtgtttgctgacgcaacccccactggttggggcattgccaccacctgtcagctcctttccgggactttcgctttccccctccctattgccacggcggaactcatcgccgcctgccttgcccgctgctggacaggggctcggctgttgggcactgacaattccgtggtgttgtcggggaagctgacgtcctttccatggctgctcgcctgtgttgccacctggattctgcgcgggacgtccttctgctacgtcccttcggccctcaatccagcggaccttccttcccgcggcctgctgccggctctgcggcctcttccgcgtcttcgccttcgccctcagacgagtcggatctccctttgggccgcctccccgcct

SEQ ID NO: 23 - RNA, 100 nt, synthetic construct
  source 1..100: mol_type = other RNA
ggacctggtggcactgaagagttttagagctagaaatagcaagttaaaataaggctagtccgttatcaacttgaaaaagtggcaccgagtcggtgctttt

SEQ ID NO: 24 - RNA, 100 nt, synthetic construct
  source 1..100: mol_type = other RNA
ttgccggcacgccaacatcggttttagagctagaaatagcaagttaaaataaggctagtccgttatcaacttgaaaaagtggcaccgagtcggtgctttt

SEQ ID NO: 25 - DNA, 1464 bp, synthetic construct
  source 1..1464: mol_type = other DNA
gccaccatggccttaccagtgaccgccttgctcctgccgctggccttgctgctccacgccgccaggccggacatccagatgacacagactacatcctccctgtctgcctctctgggagacagagtcaccatcagttgcagggcaagtcaggacattagtaaatatttaaattggtatcagcagaaaccagatggaactgttaaactcctgatctaccatacatcaagattacactcaggagtcccatcaaggttcagtggcagtgggtctggaacagattattctctcaccattagcaacctggagcaagaagatattgccacttacttttgccaacagggtaatacgcttccgtacacgttcggaggggggaccaagctggagatcacaggtggcggtggctcgggcggtggtgggtcgggtggcggcggatctgaggtgaaactgcaggagtcaggacctggcctggtggcgccctcacagagcctgtccgtcacatgcactgtctcaggggtctcattacccgactatggtgtaagctggattcgccagcctccacgaaagggtctggagtggctgggagtaatatggggtagtgaaaccacatactataattcagctctcaaatccagactgaccatcatcaaggacaactccaagagccaagttttcttaaaaatgaacagtctgcaaactgatgacacagccatttactactgtgccaaacattattactacggtggtagctatgctatggactactggggtcaaggaacctcagtcaccgtctcctcaaccacgacgccagcgccgcgcccaccaacaccggcgcccaccatcgcgtcgcagcccctgtccctgcgcccagaggcgtgccggccagcggcggggggcgccgtgcacacgagggggctggacttcgcctgtgatatctacatctgggcgcccctggccgggacctgtggggtcctgctcctgagcctggtgatcaccctgtactgcaagcggggccgcaagaagctcctgtacatcttcaagcagccatttatgcgcccagtgcagaccacccaggaggaggatggctgctcgtgccgcttcccagaggaggaagagggcggctgtgagctgcgcgtgaagttcagcaggagcgccgacgcccccgcgtacaagcagggccagaaccagctctacaacgagctcaacctgggccgccgcgaggagtacgatgtgctggacaagcgccgcggccgggaccctgagatggggggcaagccgcgcaggaagaaccctcaggagggcctgtacaacgagctgcagaaggataagatggcggaggcctacagcgagatcgggatgaagggcgagcgccggaggggcaaggggcacgatggcctgtaccagggcctcagcacagccaccaaggacacctacgacgccctgcacatgcaggccctgccccctcgc

SEQ ID NO: 26 - DNA, 47 bp, synthetic construct
  source 1..47: mol_type = other DNA
  modified_base 1..5: mod_base = OTHER; note = Phosphorothioated
taataccatcttcagtgccaccaggtcccctgcgcagtgcagaggag

SEQ ID NO: 27 - DNA, 29 bp, synthetic construct
  source 1..29: mol_type = other DNA
caactgagagaactcaaaggttaccccag

SEQ ID NO: 28 - DNA, 47 bp, synthetic construct
  source 1..47: mol_type = other DNA
  modified_base 1..5: mod_base = OTHER; note = Phosphorothioated
taataggacctggtggcactgaagatggcctgcgcagtgcagaggag

SEQ ID NO: 29 - DNA, 48 bp, synthetic construct
  source 1..48: mol_type = other DNA
taataggacctggtggcactgaagatggggtcagaaatgggaatttta